(19) United States
(12) Patent Application Publication
(10) Pub. No.: US 2013/0233326 A1
(43) Pub. Date: Sep. 12, 2013

(54) FLOATING WOUND PADS

(71) Applicant: Samuel L. Morris, III, Buffalo, NY (US)

(72) Inventor: Samuel L. Morris, III, Buffalo, NY (US)

(73) Assignee: MILSPORT MEDICAL PRODUCTS, LLC, Buffalo, NY (US)

(21) Appl. No.: 13/637,807

(22) PCT Filed: Apr. 1, 2011
(86) PCT No.: PCT/US2011/030880

§ 371 Date: May 31, 2013

Related U.S. Application Data
(60) Provisional application No. 61/321,741, filed on Apr. 7, 2010.

(51) Int. Cl. A61F 15/00; B65D 81/02

(52) U.S. Cl. CPC A61F 15/008; B65D 81/02
USPC 128/888; 428/76

(57) ABSTRACT

The present invention relates to a device comprising, a fixed surface having a means of adhering on a protective covering for shielding a body part or a fragile object, an at least one pad adapted to fit on the fixed surface and, an exterior cover substantially enclosing said pad, the exterior cover having a means of adhering on one surface, wherein the means of adhering of the fixed surface contacts the means of adhering of the exterior cover enclosing the pad and the exterior cover enclosing the pad can be detached, moved, and contacted on any location of the fixed surface.

### REFERENCE TO CO-PENDING APPLICATIONS

Priority is claimed as a 371 of international application number PCT/IL2011/030880, filed on Apr. 1, 2011; which claims priority to U.S. Provisional Patent application Ser. No. 61/321,741, filed on Apr. 7, 2010.

The present invention relates to the field of protective covering for shielding a body part or an object.

Protective coverings often have padding useful for protecting a wearer's body parts from injury. Protective coverings are also used for shipping fragile objects. Most commonly, these protective pads are made to include an outer shell that is hard and an inner shell that is soft. Such devices are typically strapped to a desired degree of tightness around the body part or object requiring protection, e.g., a wearer's elbow, knee, shin, etc. Accordingly, the interior of the devices are usually cup-shaped so as to comfortably receive the body part requiring protection. These protective coverings are fixed in place and do not accommodate differences in the shape of various parts of a body or objects. The pads found in protective coverings currently available do not adjust to irregular wound sites or irregularly shaped objects.

Therefore, a need remains for an improved protective covering device that has pads which to fit a wearer's body part even when a wound is present or accommodate an irregularly shaped object.

### SUMMARY OF THE INVENTION

According to a broad aspect, the invention seeks to provide a device, comprising a fixed surface attached to a protective covering for shielding a body part, an at least one enclosed pad comprising a pad having an exterior cover, the exterior cover having a means of adhering on a part of the exterior cover but not on the entire exterior cover, and the means of adhering of the fixed surface contacts the means of adhering of the pad, and the pad can be detached, moved, and contacted on any location of the fixed surface.

According to another broad aspect, the invention seeks to provide a device, comprising, a fixed surface attached to a shipping container, an at least one enclosed pad comprising a pad having an exterior cover, the exterior cover having a means of adhering on a part of the exterior cover but not on the entire exterior cover, and the means of adhering of the fixed surface contacts the means of adhering of the pad, and the pad can be detached, moved, and contacted on any location of the fixed surface.

For purpose of this invention the pad has a thickness of about 1.50 mm to about 75.0 mm and the pad is made of a material that provides a yieldable resilient interior having a consistent memory. Examples include, but are not limited to, an open cell or close cell foam material, an inflatable material, polyurethane, a combination of polymers, and a combination of co-polymers. The pad can be removed from the fixed surface and washed.

The pad is covered with an exterior cover substantially enclosing the pad (enclosed pad). The exterior cover has a means of adhering on a part of the exterior cover but not the entire cover. The overall material of the exterior cover is flexible. The exterior cover may consist only of a means of adhering in which case the exterior cover does not enclose the pad but instead is fixed to some portion of the pad.

The device can serve many functions including the following non-limiting functions; an orthotic, a bandage, a pad that is movable to surround a wound and is made to subjectively fit any body part.

In a non-limiting embodiment of this invention the fixed surface is attached to the protective covering with adhesive, stitching or snaps.

In a non-limiting embodiment the protective covering has a body contacting inner side and an outer side, and a plurality of fastening elements affixed to said outer side. The fixed surface optionally has an opening to accommodate a wound.

In a non-limiting embodiment the pads are movable to support an object within the shipping container and the fixed surface is fixed to the shipping container with an adhesive. The shipping container can be, for example, a carrying device such as a backpack and the movable pads can be placed on the straps of the carrying device.

In a non-limiting embodiment the means of adhering is a hook texture, a loop texture, or a combination of hook and loop texture. In another embodiment the means of adhering can be a non-drying adhesive.

In a non-limiting embodiment the fixed surface can optionally have an exterior cover independent of the pad covering all or part of the fixed surface.

### BRIEF DESCRIPTION OF THE DRAWINGS

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

FIG. 1 is a perspective view of a device configured and arranged in accordance with an implementation of the invention.

FIG. 2 is the device in FIG. 1 used in association with a shipping container or protective covering.

### DETAILED DESCRIPTION

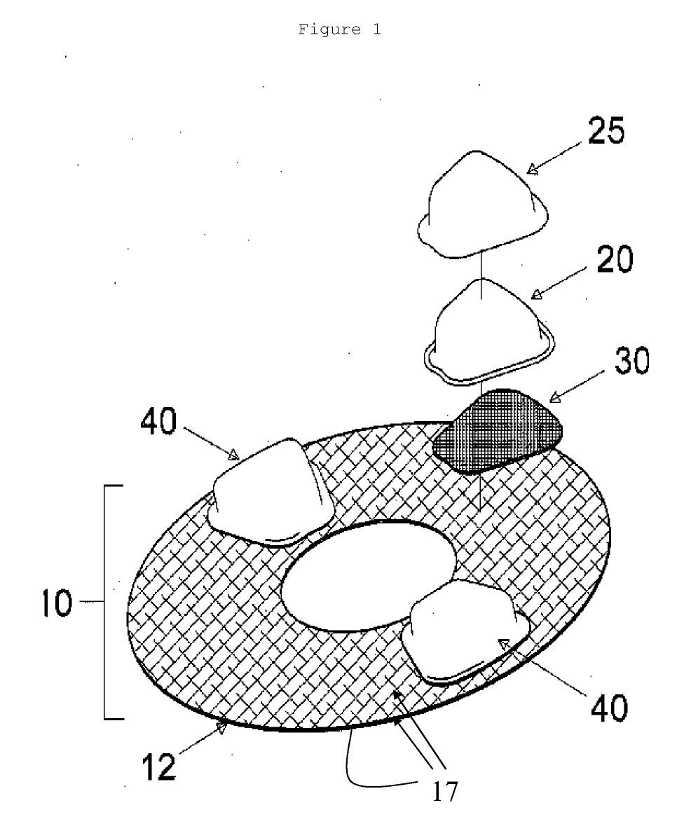

FIG. 1 shows an example of the device 10 made in accordance with the invention. The device 10 includes a fixed surface 12. The fixed surface 12 (a) is or (b) is covered by a first means of adhering 17. At least one pad 20 has an exterior cover 25 enclosing the pad 20. A second means of adhering 30 is located on the exterior cover but is not part of the entire cover. The enclosed pad 40 includes the exterior cover 25, the pad 20, and the means of adhering 30 and can be detached, moved, and contacted at any location of the fixed surface 12.

A body or an object needs protection from trauma during recovery from an injury, performing a sports activity, or in the process of moving from one location to another. A body is considered the entire material or physical structure of an organism or object, especially of a human or an animal. A body part is any portion of the body.

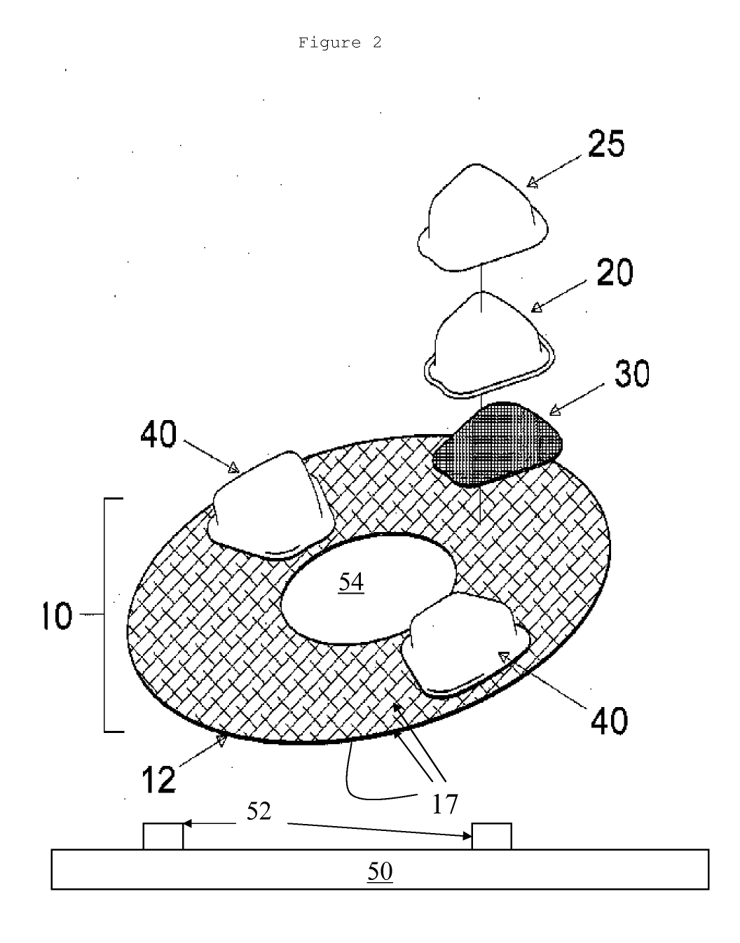

A means of protection for the body or object is in the form of a protective covering or shipping container 50, as illustrated in FIG. 2. The protective covering or shipping container 50 can be hard or soft and often requires a cushion to surround the body or object. The at least one enclosed pads 40 (also referred to as pad 40) can be one pad 40 or any number of pads needed to provide a cushion or a source of pressure application on the interior of a protective covering or shipping container 50. The pad 40 can be any shape or size needed to serve as a cushion like mass of soft material used for protection. The pad 20 has a thickness of about 1.50 mm to about 75.0 mm and the pad 40 is made of a material that provides a yieldable resilient interior having a consistent memory. Examples include, but are not limited to, an open cell or close cell foam material, an inflatable material, polyurethane, a combination of polymers, and a combination of copolymers. The pad 40 material can be removed from the fixed surface 12 and washed.

The pad 40 has an exterior cover 25. The exterior cover 25 occupies some portion of the surface of the pad 40. The exterior cover 25 is an additional part that is spread over, envelops, or coats the surface of the pad 20. The exterior cover 25 provides a way to attach the pad 40 to a part of the fixed surface 12.

The pad 40 and the fixed surface 12 adhere to each other in a way that allows the pad 40 to be removed from one location on the fixed surface 12 to another location on the fixed surface 12 or removed from the fixed surface 12 altogether. For use in any device 10 of this invention at least one pad 40 must be adhered to the fixed surface 12. The second means of adhering 30 is not on every bit of the exterior cover 25. On the fixed surface 12 the first means of adhering 17 covers the complete surface and no part is left out. The fixed surface 12 may have the exterior cover 25 with no pad 20 therein in places where no pad 40 is adhered to the fixed surface 12.

The fixed surface 12 is securely fastened and non-removable from the protective covering or shipping container 50. The fixed surface 12 is located on the interior of the protective covering or shipping container and faces the body part or object. The fixed surface 12 is composed of a material having adhesive properties 17.

The device 10 subjectively fits any body part which means it is generally conformable to anatomical surfaces (shielding). As such, it conforms to the surface even when the surface is moved even when a joint is flexed and then returned to its un-flexed position.

The shipping container 50 is a receptacle for holding items. The shipping container 50 may be composed of a variety of materials and may be for example metal, plastic, wood, cardboard, fabric, or a combination of materials.

The fixed surface 12 may be attached to the protective covering 50 by snaps, buttons, adhesive, stitching, or any other permanent attachment device 52. When an adhesive is used it may be a low adhesion coating (adhesive). The adhesive can be single sided or double sided. When the adhesive is double sided the adhesive is characterized as adhering to the protective covering 50 and the enclosed pad or an exterior cover.

The first and second means of adhering 17, 30 can have a hooked and/or loop texture, for example, but not limited to VELCRO, or a non-drying adhesive surface. The first and second means of adhering 17, 30 has the characteristic of adhering more then one time without loss of texture or adhesive character.

A wound is any body part that has experienced a trauma. The trauma can manifest as an ulcer, a bedsore, a blister, an incision, a burn, a fistula, a fracture, or a hematoma. A trauma can also be internal (i.e. torn ligament) in which case the device can serve to apply pressure. The protective covering 50 and the fixed surface 12 can have an open area 54 that allows the wound to be exposed to air circulation. The enclosed pads 40 attached to the fixed surface 12 elevate the wound away from the surface of the protective covering thereby offering protection to the wound while still allowing air circulation to the wound.

1. A device comprising:
a fixed surface (a) for inhibiting further damage to a wound on a patient's body part, (b) has an exterior distal surface and an exterior proximal surface in relation to the patient's wound, (c) has an adherable surface on the proximal exterior surface;
at least one enclosed pad comprising a pad enveloped by an exterior cover;
the exterior cover includes an adhering material on a part of the exterior cover but not on the entire exterior cover; and
the adhering material contacts the adherable surface, and the enclosed pad can be detached, moved, and contacted on any location of the fixed surface's exterior proximal surface to (a), depending on the desired therapy, apply pressure to the wound or not apply pressure to the wound and (b) separate the patient's body from the exterior proximal surface.

2. The device of claim 1, wherein the pad has a thickness of about 1.50 mm to about 75.0 mm.

3. The device of claim 1, wherein the pad is a foam material.

4. The device of claim 3, wherein the foam material is selected from the group consisting of an open cell foam material, close cell foam material, polyurethane, a combination of polymers, and a combination of copolymers.

5. The device of claim 1, wherein the interior of the pad is an inflatable material.

6. The device of claim 1, wherein the pads are yieldable resilient and has a consistent memory.

7. The device of claim 1, wherein the exterior cover is flexible.

8. The device of claim 1, wherein the exterior distal surface attaches to a protective covering and the protective covering is an orthotic.

9. The device of claim 1, wherein the exterior distal surface attaches to a protective covering and the protective covering is a bandage.

10. The device of claim 1, wherein the at least one enclosed pad is movable to surround the wound.

11. The device of claim 1, wherein the device subjectively fits any body part.

12. The device of claim 1, wherein the fixed surface has an opening to expose the wound.

13. The device of claim 1, wherein the fixed surface's exterior distal surface attaches to a protective covering with an adhesive.

14. The device of claim 1, wherein the fixed surface's exterior distal surface attaches to a protective covering with stitching.

15. The device of claim 1, wherein the fixed surface's exterior distal surface attaches to a protective covering with snaps.

16. The device of claim 1, wherein the fixed surface's exterior distal surface attaches to a protective covering and the protective covering (a) shields the body part, (b) has a body contacting inner side and an outer side, and (c) has a plurality of fastening elements affixed to said inner side.

17. A device comprising:
a fixed surface (a) for inhibiting damage to an object in a shipping container, (b) has an exterior distal surface and an exterior proximal surface in relation to the patient's wound, (c) has an adherable surface on the proximal exterior surface;
at least one enclosed pad comprising a pad enveloped by an exterior cover;
the exterior cover includes an adhering material on a part of the exterior cover but not on the entire exterior cover; and
the adhering material contacts the adherable surface, and the enclosed pad can be detached, moved, and contacted on any location of the fixed surface to separate the object from the fixed surface's exterior proximal surface and support the object within the shipping container.

18. (canceled)

19. The device of claim 17, wherein the fixed surface's exterior distal surface attaches to the shipping container with an adhesive.

20. The device of claim 1, wherein the adhering material and the adherable surface are selected from the group consisting of a hook texture, a loop texture, or a combination of hook and loop texture.

21. The device of claim 1, wherein the adhering material is a non drying adhesive.

22. (canceled)
